(19) United States
(12) Patent Application Publication
(10) Pub. No.: US 2020/0214630 A1
(43) Pub. Date: Jul. 9, 2020

(54) Psychological Pressure Evaluation Method and Device

(71) Applicant: Huawei Technologies Co., Ltd., Shenzhen (CN)

(72) Inventors: Lan Xia, Shenzhen (CN); Zhengkui Liu, Beijing (CN); Fenghua Li, Beijing (CN); Shichun Zheng, Beijing (CN); Hongrui Jiang, Shenzhen (CN); Peida Xu, Shenzhen (CN); Anqi Zhang, Shenzhen (CN)

(21) Appl. No.: 16/640,858

(22) PCT Filed: Aug. 24, 2017
(86) PCT No.: PCT/CN2017/098896

§ 371 Date: Feb. 21, 2020

(51) Int. Cl. A61B 5/00; A61B 5/0488; A61B 5/0476; A61B 5/0205; A61B 5/16; G16H 20/70; G16H 50/30

(52) U.S. Cl. CPC A61B 5/4884; A61B 5/0488; A61B 5/0476; A61B 5/0205; A61B 5/024; A61B 5/7264; A61B 5/7275; G16H 20/70; G16H 50/30; A61B 5/165

(57) ABSTRACT

A psychological pressure evaluation method includes obtaining a physiological parameter measurement value of a user, determining a psychological pressure evaluation model of the user based on a physiological parameter reference value of the user and a common psychological pressure evaluation model, where the common psychological pressure evaluation model reflects a change relationship between a psychological pressure value change amount and a physiological parameter value change amount, and determining a psychological pressure evaluation result of the user based on the physiological parameter measurement value of the user and the psychological pressure evaluation model of the user.

### TECHNICAL FIELD

This application relates to the terminal field, and in particular, to a psychological pressure evaluation method and device.

### BACKGROUND

An intrapsychic conflict of a person and emotion experience accompanied with the intrapsychic conflict are pressure in psychology, are referred to as psychological pressure, and are also referred to as mental pressure. The psychological pressure is cognition and behavior experience that includes both a pressure source and a pressure reaction.

Modern medicine proves that the psychological pressure may weaken a human body immunity system, and consequently, an external pathogenic factor causes a disease. When the psychological pressure exceeds psychological endurance of a human body, psychological imbalance occurs, and a psychological disease such as depression or anxiety is caused.

In conventional psychology, for psychological pressure evaluation, a subjective self-evaluation method such as a measurement table or a questionnaire and face-to-face conversation and observation of a professional psychologist need to be combined. Therefore, this method is not suitable as a feasible solution to continuously evaluate psychological pressure in real time.

In the prior art, a method for establishing a psychological pressure evaluation model is further provided, and may be used to evaluate psychological pressure in real time. Specifically, it is assumed that n testers participate in a psychological pressure stimulus experiment. A subjective psychological pressure self-evaluation value of each tester after the experiment is obtained, and \vec{Y}=(y₁, y₂, . . . yᵢ, . . . yₙ). In addition, a physiological parameter measurement value of each tester is obtained through detection by using a sensor in an experiment process, and \vec{X}=(x₁, x₂, . . . , xᵢ, . . . , xₙ). Therefore, a total of n data samples corresponding to the n testers are obtained. i represents a sequence number of a sample, xᵢ represents a physiological parameter measurement value in a sample i, and yᵢ represents a subjective psychological pressure self-evaluation value, namely, a value obtained after an i^{th} tester evaluates a psychological pressure feeling generated in the psychological pressure stimulus experiment, in the sample i. A training data set (\vec{X}, \vec{Y}) of the model is established. A machine learning method is designed, \vec{X} is used as an input of an algorithm, and \vec{Y} is used as an output of the algorithm, to establish a model \vec{Y}=ƒ(\vec{X}) between \vec{X} and \vec{Y}.

However, due to particularity of a psychological question, and a difference between cognition levels of persons for their bodies, subjective psychological pressure self-evaluation values of all testers have different accuracy. In other words, some data in (y₁, y₂, . . . , yᵢ, . . . , yₙ) is inaccurate. Therefore, directly using all data for modeling likely introduces noise into the training data set. Consequently, the established model is inaccurate. It is clear that psychological pressure of a user that is evaluated by using the model has relatively poor reliability because the model is inaccurate.

### SUMMARY

This application provides a psychological pressure evaluation method and device, to resolve a problem that psychological pressure of a user that is evaluated by using an existing psychological pressure evaluation model has relatively poor reliability.

According to a first aspect, this application provides a psychological pressure evaluation method, including: obtaining a physiological parameter measurement value of a user; and determining a psychological pressure evaluation result of the user based on the physiological parameter measurement value of the user and a psychological pressure evaluation model of the user. The psychological pressure evaluation model of the user is determined based on a physiological parameter reference value of the user, a psychological pressure value corresponding to the physiological parameter reference value of the user, and a common psychological pressure evaluation model, or the psychological pressure evaluation model of the user is determined based on a physiological parameter reference value of the user and a common psychological pressure evaluation model. The common psychological pressure evaluation model reflects a change relationship between a psychological pressure value change amount and a physiological parameter value change amount. The physiological parameter value change amount is a difference between a physiological parameter measurement value and a physiological parameter reference value. The psychological pressure value change amount is a difference between a psychological pressure value corresponding to the physiological parameter measurement value and a psychological pressure value corresponding to the physiological parameter reference value.

Therefore, according to the method, a psychological pressure evaluation device can perform long-term continuous, non-perceptive, and real-time psychological pressure evaluation on the user, to improve reliability of psychological pressure prediction of the user.

In a possible design, the common psychological pressure evaluation model is obtained based on a training data set by using a preset machine learning method. The training data set includes a plurality of training data groups. An i^{th} training data group includes an i^{th} physiological parameter value change amount and an i^{th} psychological pressure value change amount. The i^{th} physiological parameter value change amount is a difference between a physiological parameter measurement value of an i^{th} tester and a physiological parameter reference value of the i^{th} tester. The i^{th} psychological pressure value change amount is a difference between a psychological pressure value corresponding to the physiological parameter measurement value of the i^{th} tester and a psychological pressure value corresponding to the physiological parameter reference value of the i^{th} tester.

Therefore, the common psychological pressure evaluation model can be used to eliminate a subjective factor of psychological pressure evaluation to some extent, so that a modeling result more objectively and accurately reflects a psychological pressure value change. In addition, in the common psychological pressure evaluation model obtained according to the foregoing modeling method, when the training data set is interfered by a subjective factor of the user, a training data group loss can be avoided, and impact of noise data in the training data set in a modeling process can be effectively avoided. This ensures that the established model between a psychological pressure value and a physiological parameter measurement value is more accurate.

In a possible design, when the psychological pressure evaluation result of the user is determined based on the physiological parameter measurement value of the user and the psychological pressure evaluation model of the user, a psychological pressure value corresponding to the physiological parameter measurement value of the user may be specifically determined based on the physiological parameter measurement value of the user and the psychological pressure evaluation model of the user. The psychological pressure evaluation model of the user is determined based on the physiological parameter reference value of the user, the psychological pressure value corresponding to the physiological parameter reference value of the user, and the common psychological pressure evaluation model.

Therefore, the psychological pressure evaluation model provided for the user in this application is personalized, and can provide a relatively accurate psychological pressure evaluation result for the user.

In a possible design, when the psychological pressure evaluation result of the user is determined based on the physiological parameter measurement value of the user and the psychological pressure evaluation model of the user, a psychological pressure value change amount of the user may be specifically determined based on the physiological parameter measurement value of the user and the psychological pressure evaluation model of the user. The psychological pressure evaluation model of the user is determined based on the physiological parameter reference value of the user and the common psychological pressure evaluation model.

Therefore, the psychological pressure evaluation model provided for the user in this application is personalized, and can provide a relatively accurate psychological pressure evaluation result for the user.

In a possible design, before the obtaining a physiological parameter measurement value of a user, whether the psychological pressure evaluation model of the user is stored is queried. When it is determined that the psychological pressure evaluation model of the user is not stored, the physiological parameter reference value of the user is obtained, and the psychological pressure value corresponding to the physiological parameter reference value of the user is determined. A difference between a moment of obtaining the physiological parameter reference value of the user and a moment of determining the psychological pressure value corresponding to the physiological parameter reference value of the user is less than or equal to preset duration. Then, the psychological pressure evaluation model of the user is determined based on the physiological parameter reference value of the user, the psychological pressure value corresponding to the physiological parameter reference value of the user, and the common psychological pressure evaluation model.

Therefore, before the user starts psychological pressure measurement, whether the psychological pressure evaluation model of the user is stored is first queried. When it is determined that the psychological pressure evaluation model of the user is not stored, the psychological pressure evaluation model of the user needs to be established. In addition, the difference between the moment of obtaining the physiological parameter reference value of the user and the moment of determining the psychological pressure value corresponding to the physiological parameter reference value of the user is less than or equal to the preset duration. Therefore, accuracy and validity of the psychological pressure evaluation model of the user can be ensured.

In a possible design, before the obtaining a physiological parameter measurement value of a user, whether the psychological pressure evaluation model of the user is stored is queried. When it is determined that the psychological pressure evaluation model of the user is not stored, the physiological parameter reference value of the user is obtained, and the psychological pressure evaluation model of the user is determined based on the physiological parameter reference value of the user and the common psychological pressure evaluation model.

Therefore, before the user starts psychological pressure measurement, whether the psychological pressure evaluation model of the user is stored is first queried. When it is determined that the psychological pressure evaluation model of the user is not stored, the psychological pressure evaluation model of the user needs to be established.

In a possible design, the psychological pressure value corresponding to the physiological parameter reference value of the user is a subjective psychological pressure self-evaluation value of the user. The subjective psychological pressure self-evaluation value of the user is obtained in at least one of the following manners; a manner in which the user answers a preset psychological pressure questionnaire, a manner in which the user scores a preset psychological pressure question, or a manner of performing automatic analysis and recognition by using a preset event or preset behavior.

Therefore, this application provides a plurality of manners for obtaining the subjective psychological pressure self-evaluation value of the user.

In a possible design, after the determining a psychological pressure evaluation result of the user, the method further includes: prompting the user when the psychological pressure evaluation result of the user meets a preset psychological pressure prompt condition.

In a possible design, when the psychological pressure value corresponding to the physiological parameter measurement value of the user is greater than a preset first threshold, the user is prompted.

In a possible design, when the psychological pressure value change amount of the user is greater than a preset second threshold, the user is prompted.

Therefore, in this application, when the psychological pressure evaluation result of the user meets the preset psychological pressure prompt condition, the user can be prompted in a timely manner, so that the user can adjust psychological pressure in a timely manner when no body damage is caused by the psychological pressure, and can use a proper pressure-release means based on a long-term change status of the psychological pressure, to keep healthy.

According to a second aspect, this application provides a psychological pressure evaluation device, including a physiological parameter measurement device, a processor, and a memory. The physiological parameter measurement device is configured to measure a physiological parameter of a user, to obtain a physiological parameter measurement value of the user. The memory is configured to store a program, a psychological pressure evaluation model of the user, and a common psychological pressure evaluation model. The processor is configured to invoke the program stored in the memory, to perform the method in the first aspect. Optionally, the psychological pressure evaluation device further includes a display, and the display is configured to display a psychological pressure evaluation result of the user.

According to a third aspect, this application provides a psychological pressure evaluation device, including a communications interface, a processor, and a memory. The communications interface is configured to receive a physiological parameter measurement value of a user that is sent by an external physiological parameter measurement device. The memory is configured to store a program, a psychological pressure evaluation model of the user, and a common psychological pressure evaluation model. The processor is configured to invoke the program stored in the memory, to perform the method in the first aspect. Optionally, the psychological pressure evaluation device further includes a display, and the display is configured to display a psychological pressure evaluation result of the user.

According to a fourth aspect, this application further provides a computer storage medium storing a computer executable instruction, where when the computer executable instruction is run on a computer, the computer is enabled to perform the method in the first aspect of this application.

According to a fifth aspect, this application further provides a computer program product, where the computer program product includes the computer executable instruction stored in the foregoing computer storage medium, and when the computer executable instruction is run on a computer, the computer is enabled to perform the method in the first aspect of this application.

### BRIEF DESCRIPTION OF DRAWINGS

FIG. 1 is a schematic diagram of establishing a common psychological pressure evaluation model according to this application;

FIG. 2 is a first schematic structural diagram of a psychological pressure evaluation device according to this application;

FIG. 3 is a second schematic structural diagram of a psychological pressure evaluation device according to this application;

FIG. 4 is a first specific flowchart of evaluating psychological pressure of a user by using a psychological pressure evaluation device according to this application; and

FIG. 5 is a second specific flowchart of evaluating psychological pressure of a user by using a psychological pressure evaluation device according to this application.

### DESCRIPTION OF EMBODIMENTS

The following describes the embodiments of this application with reference to accompanying drawings.

This application may be applied to a scenario in which psychological pressure is evaluated by measuring a physiological parameter, for example, a mobile phone, a watch, a wristband, glasses, and a medical professional device.

In a modeling process of a common psychological pressure evaluation model, there may be a plurality of physiological parameters, such as an electrocardiograph, an electromyography, a pulse wave, and an electroencephalogram. A psychological pressure stimulus experiment is to perform psychological stimulus on a tester by using a means such as sound, a picture, or a video. Further, in an experiment process, a physiological parameter of the tester may be measured by using a sensor. After the experiment is completed, a subjective psychological pressure self-evaluation value of the tester is collected. Specifically, a manner for obtaining the subjective psychological pressure self-evaluation value may be that the tester answers a psychological pressure questionnaire for the experiment process, the tester directly scores a preset psychological pressure question for the experiment process, or the like. The subjective psychological pressure self-evaluation value may be represented as a specific value or a psychological pressure level.

Compared with an existing method for establishing a psychological pressure evaluation model, in the modeling process of the common psychological pressure evaluation model in this application, before a physiological parameter measurement value of the tester and a psychological pressure value corresponding to the physiological parameter measurement value are obtained, a physiological parameter reference value of the tester and a psychological pressure value corresponding to the physiological parameter reference value need to be first obtained as baseline data. A change amount from a physiological parameter value before the experiment to a physiological parameter value after the experiment is used as an input of an algorithm, and a change amount from a psychological pressure value before the experiment to a psychological pressure value after the experiment is used as an output of the algorithm.

It should be understood that, in the modeling process of the common psychological pressure evaluation model, both the psychological pressure value corresponding to the physiological parameter measurement value and the psychological pressure value corresponding to the physiological parameter reference value are subjective psychological pressure self-evaluation values of the tester. Although a one-time absolute psychological pressure self-evaluation value of the tester may be inaccurate, a measurement criterion of the tester for subjective cognition of the tester within a short time may be considered to be unchanged. Therefore, the change amount from the psychological pressure value before the experiment to the psychological pressure value after the experiment can accurately reflect a psychological pressure change of the tester, and is not affected by accuracy of the one-time subjective psychological pressure self-evaluation value.

For example, if a tester has an inherent offset A in cognition of psychological pressure of the tester, and a real psychological pressure value before the psychological pressure stimulus experiment is B, a subjective psychological pressure self-evaluation value provided by the tester before the psychological pressure stimulus experiment is A+B. If the inherent offset A of the tester is unchanged within a short time, and the real psychological pressure value after the psychological pressure stimulus experiment changes to C, a subjective psychological pressure self-evaluation value provided by the tester after the psychological pressure stimulus experiment is A+C. A change amount from the subjective psychological pressure self-evaluation value before the experiment to the subjective psychological pressure self-evaluation value after the experiment is (A+C)−(A+B), and can correctly reflect a change amount C−B from the real psychological pressure value before the stimulus experiment to the real psychological pressure value after the stimulus experiment.

A training data set of the common psychological pressure evaluation model includes a plurality of training data groups. An i^{th} training data group includes an i^{th} physiological parameter value change amount and an i^{th} psychological pressure value change amount. The i^{th} physiological parameter value change amount is a difference between a physiological parameter measurement value of an i^{th} tester and a physiological parameter reference value of the i^{th} tester. The i^{th} psychological pressure value change amount is a difference between a psychological pressure value corresponding to the physiological parameter measurement value of the i^{th} tester and a psychological pressure value corresponding to the physiological parameter reference value of the i^{th} tester.

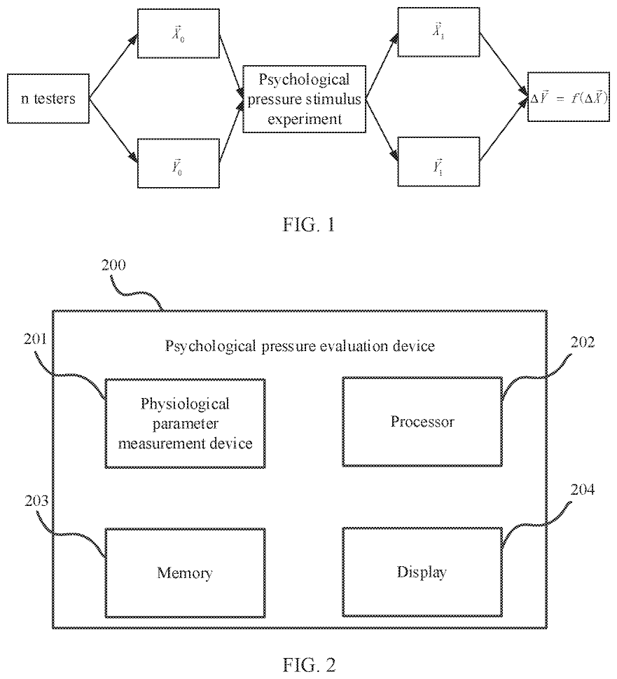

As shown in FIG. 1, specifically, physiological parameter reference values of n testers are represented by \vec{X}₀, and \vec{X}₀=(x₁⁰, x₂⁰, . . . , xᵢ⁰, . . . , xₙ⁰). Psychological pressure values of the n testers that are corresponding to the physiological parameter reference values of the n testers are represented by \vec{Y}₀, and \vec{Y}₀=(y⁰₁, y⁰₂, . . . , y⁰ᵢ, . . . , y⁰ₙ). \vec{X}₀ and \vec{Y}₀ are obtained before a psychological pressure stimulus experiment. After the psychological pressure stimulus experiment, physiological parameter measurement values of the n testers and psychological pressure values of the n testers that are corresponding to the physiological parameter measurement values of the n testers are collected. The physiological parameter measurement values of the n testers are represented by \vec{X}₁, and \vec{X}₁=(x₁¹, x¹₂, . . . , x¹ᵢ, . . . , x¹ₙ). The psychological pressure values of the n testers that are corresponding to the physiological parameter measurement values of the n testers are represented by \vec{Y}₁, and \vec{Y}₁=(y¹₁, y¹₂, . . . , y¹ᵢ, . . . , y¹ₙ). In this case, a physiological parameter value change amount is Δ\vec{X}=\vec{X}₁−\vec{X}₀, and a psychological pressure value change amount is Δ\vec{Y}=\vec{Y}₁−\vec{Y}₀. n is a positive integer, and is usually a relatively large number.

Further, a common psychological pressure evaluation model Δ\vec{Y}=ƒ(Δ\vec{X}) is established by using a machine learning method. Therefore, the common psychological pressure evaluation model can be used to eliminate a subjective factor of psychological pressure evaluation to some extent, so that a modeling result more objectively and accurately reflects a psychological pressure value change. A specific machine learning method may be a classification algorithm or a fitting algorithm. This application does not relate to improvement on a specific algorithm. Details are not described herein.

In addition, in the common psychological pressure evaluation model obtained according to the foregoing modeling method, when a training data set is interfered by a subjective factor of a user, a training data group loss can be avoided, and impact of noise data in the training data set in a modeling process can be effectively avoided. This ensures that the established model between a psychological pressure value and a physiological parameter measurement value is more accurate.

After the common psychological pressure evaluation model is obtained, and before psychological pressure evaluation is performed on the user, a psychological pressure evaluation model of the user, namely, a personalized psychological pressure evaluation model, further needs to be determined.

Specifically, the psychological pressure evaluation model of the user may have two types.

A first type is as follows: The psychological pressure evaluation model of the user is determined based on a physiological parameter reference value of the user, a psychological pressure value corresponding to the physiological parameter reference value of the user, and the common psychological pressure evaluation model.

Because physiological parameter reference values of all user are different, and psychological pressure values corresponding to the physiological parameter reference values of the users are different, psychological pressure evaluation models of the users are also different. Therefore, the psychological pressure evaluation model provided for the user in this application is personalized, and can provide a relatively accurate psychological pressure evaluation result for the user.

Specifically, similar to that in the foregoing modeling process, the physiological parameter reference value of the user may be measured by using a sensor, and the psychological pressure value corresponding to the physiological parameter reference value of the user may be obtained in at least one of the following manners:

a manner in which the user answers a preset psychological pressure questionnaire, a manner in which the user scores a preset psychological pressure question, or a manner of performing automatic analysis and recognition by using a preset event or preset behavior.

In addition, a difference between a moment of obtaining the physiological parameter reference value of the user and a moment of determining the psychological pressure value corresponding to the physiological parameter reference value of the user is less than or equal to preset duration. The preset duration herein is usually relatively short, to ensure accuracy and validity of the psychological pressure evaluation model of the user.

For example, the physiological parameter reference value of the user is represented by x₀, the psychological pressure value corresponding to the physiological parameter reference value of the user is represented by y₀, and the psychological pressure evaluation model of the user is obtained with reference to the common psychological pressure evaluation model:

y=ƒ(x−x₀)+y₀,

Therefore, in the first type of psychological pressure evaluation model of the user, a physiological parameter measurement value of the user is used as an independent variable, and a psychological pressure value corresponding to the physiological parameter measurement value of the user is used as a dependent variable.

A second type is as follows: The psychological pressure evaluation model of the user is determined based on a physiological parameter reference value of the user and the common psychological pressure evaluation model.

Because physiological parameter reference values of all users are different, psychological pressure evaluation models of the users are also different. Therefore, the psychological pressure evaluation model provided for the user in this application is personalized, can provide a relatively accurate psychological pressure evaluation result for the user, and can avoid interference caused by a subjective factor.

For example, the physiological parameter reference value of the user is represented by x₀, and the psychological pressure evaluation model of the user is obtained with reference to the common psychological pressure evaluation model:

y=ƒ(x−x₀).

Therefore, in the second type of psychological pressure evaluation model of the user, a physiological parameter measurement value of the user is used as an independent variable, and a psychological pressure value change amount of the user is used as a dependent variable.

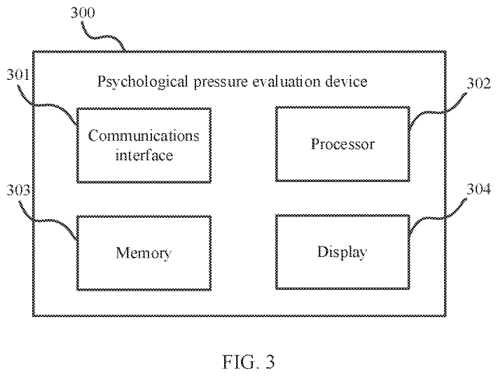

Further, the common psychological pressure evaluation model and the psychological pressure evaluation model of the user that are obtained by using the foregoing methods are stored in a memory of a psychological pressure evaluation device. FIG. 2 is a first schematic structural diagram of a psychological pressure evaluation device. The psychological pressure evaluation device includes a physiological parameter measurement device 201, a processor 202, and a memory 203. Optionally, the psychological pressure evaluation device further includes a display 204, and a power supply and another communications interface (not shown).

The processor 202 may be a central processing unit (central processing unit, CPU), and the memory 203 may include a random access memory (random-access memory. RAM), and may further include a nonvolatile memory.

A possible hardware form of the psychological pressure evaluation device shown in FIG. 2 is an intelligent terminal, a wearable device, or the like.

The physiological parameter measurement device 201 may be a sensor configured to measure a physiological parameter of a user. The physiological parameter measurement device is configured to measure the physiological parameter of the user, to obtain a physiological parameter measurement value of the user.

The memory 203 is configured to: store a program, a common psychological pressure evaluation model, and a psychological pressure evaluation model of the user; and store a physiological parameter reference value of the user, a psychological pressure value corresponding to the physiological parameter reference value of the user, historical data of a psychological pressure value of the user in a previous period, and the like.

The processor 202 is configured to: invoke the program stored in the memory to perform psychological pressure evaluation; and determine a psychological pressure evaluation result of the user based on the physiological parameter measurement value of the user and the psychological pressure evaluation model of the user.

The display 204 is configured to display the psychological pressure evaluation result of the user.

Specifically, when the psychological pressure evaluation model of the user is of the first type, the processor determines, based on the physiological parameter measurement value of the user and the psychological pressure evaluation model of the user, a psychological pressure value corresponding to the physiological parameter measurement value of the user. The display displays the psychological pressure value corresponding to the physiological parameter measurement value of the user.

When the psychological pressure evaluation model of the user is of the second type, a psychological pressure value change amount of the user is determined based on the physiological parameter measurement value of the user and the psychological pressure evaluation model of the user. The display displays the psychological pressure value change amount of the user.

Alternatively, the psychological pressure evaluation result of the user may be a psychological pressure level or another possible representation manner that is determined by using a preset algorithm and based on the psychological pressure value corresponding to the physiological parameter measurement value of the user or the psychological pressure value change amount of the user.

Alternatively, the psychological pressure evaluation result of the user may be presented in a plurality of manners, for example, a preset graph or picture that describes psychological pressure. A specific presentation manner is not limited in this application.

In addition, after the processor determines the psychological pressure evaluation result of the user, when the psychological pressure evaluation result of the user meets a preset psychological pressure prompt condition, the user is prompted.

In a possible design, when the psychological pressure value corresponding to the physiological parameter measurement value of the user is greater than a preset first threshold, the user is prompted.

In a possible design, when the psychological pressure value change amount of the user is greater than a preset second threshold, the user is prompted.

A plurality of manners such as a voice, a graph, a chart, and music may be used as a specific prompting manner. This is not limited in this application.

The processor may further generate a psychological pressure change trend for a preset period (for example, one week, one month, or a user-defined period) based on a historical psychological pressure evaluation result of the user, and present the trend to the user on the display, so that the user uses a proper pressure-release means based on a long-term change status of the psychological pressure, to keep healthy.

As shown in FIG. 3, this application further provides another psychological pressure evaluation device. A difference between the psychological pressure evaluation device and the psychological pressure evaluation device shown in FIG. 2 is that the psychological pressure evaluation device shown in FIG. 3 does not include a physiological parameter measurement device. The psychological pressure evaluation device shown in FIG. 3 includes a communications interface 301, a processor 302, and a memory 303. Optionally, the psychological pressure evaluation device further includes a display 304, a power supply, and the like. Specifically, the communications interface 301 may include an interface configured to communicate with another device. The interface may be a wired interface, a wireless interface, or a combination thereof. The communications interface 301 is configured to receive a physiological parameter measurement value of a user that is sent by an external physiological parameter measurement device. A function of another component is unchanged.

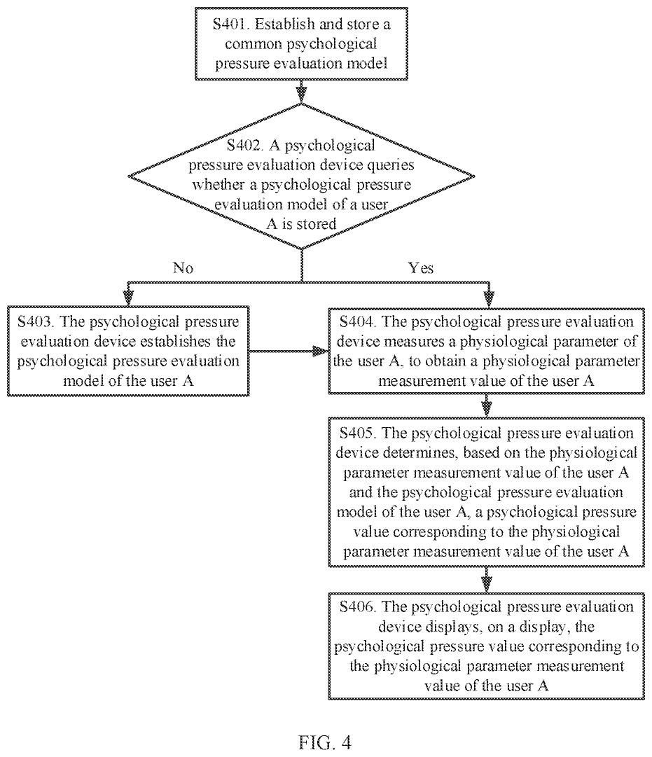

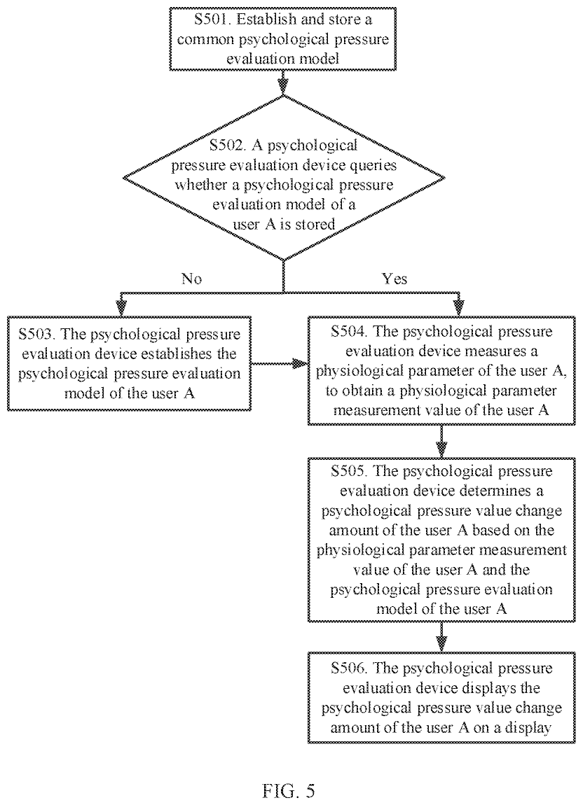

The following describes a specific implementation process of this application with reference to FIG. 4.

S401. Establish and store a common psychological pressure evaluation model.

For example, a psychological pressure stimulus experiment that covers a large quantity of samples is designed. A common psychological pressure evaluation model applicable to all people is established, and is stored in a memory of a psychological pressure evaluation device. A specific method for establishing the common psychological pressure evaluation model is described above. Details are not described herein again.

S402. The psychological pressure evaluation device queries whether a psychological pressure evaluation model of a user A is stored, and performs S403 if the psychological pressure evaluation model of the user A is not stored, or performs S404 if the psychological pressure evaluation model of the user A is stored.

For example, a display of the psychological pressure evaluation device displays names of psychological pressure evaluation models of a plurality of users that are stored in the memory, and receives a selection instruction of the user A. For example, the user A taps a touchscreen to select a name of the psychological pressure evaluation model of the user A.

S403. The psychological pressure evaluation device establishes the psychological pressure evaluation model of the user A.

Specifically, the psychological pressure evaluation device collects a physiological parameter reference value of the user A, and determines a psychological pressure value corresponding to the physiological parameter reference value of the user A. For example, the user A answers a preset psychological pressure questionnaire stored in the psychological pressure evaluation device, and the psychological pressure evaluation device determines, based on the psychological pressure questionnaire answered by the user A, the psychological pressure value corresponding to the physiological parameter reference value of the user A. Then, the psychological pressure evaluation device determines the psychological pressure evaluation model of the user A based on the physiological parameter reference value of the user A, the psychological pressure value corresponding to the physiological parameter reference value of the user A, and the common psychological pressure evaluation model.

In addition, the psychological pressure evaluation device measures the physiological parameter reference value of the user A when the psychological pressure value corresponding to the physiological parameter reference value of the user A is obtained, before the psychological pressure value corresponding to the physiological parameter reference value of the user A is obtained, or within a very short time after the psychological pressure value corresponding to the physiological parameter reference value of the user A is obtained, and stores the physiological parameter reference value in the memory of the psychological pressure evaluation device.

The psychological pressure evaluation device stores the established psychological pressure evaluation model of the user A in the memory, and continues to perform S404.

S404. The psychological pressure evaluation device measures a physiological parameter of the user A, to obtain a physiological parameter measurement value of the user A.

S405. The psychological pressure evaluation device determines, based on the physiological parameter measurement value of the user A and the psychological pressure evaluation model of the user A, a psychological pressure value corresponding to the physiological parameter measurement value of the user A.

S406. The psychological pressure evaluation device displays, on the display, the psychological pressure value corresponding to the physiological parameter measurement value of the user A.

Further, if the psychological pressure value corresponding to the physiological parameter measurement value of the user A is greater than a first preset threshold, the psychological pressure evaluation device sends a prompt to the user A, so that the user A pays attention to pressure release. The user A may further view historical data of the psychological pressure value, and adjusts a lifestyle based on a change trend of the psychological pressure value, so that the user A keeps the psychological pressure value within a normal range.

The following describes a specific implementation process of this application with reference to FIG. 5.

S501. Establish and store a common psychological pressure evaluation model.

The common psychological pressure evaluation model is stored in a memory of a psychological pressure evaluation device.

S502. The psychological pressure evaluation device queries whether a psychological pressure evaluation model of a user A is stored, and performs S503 if the psychological pressure evaluation model of the user A is not stored, or performs S504 if the psychological pressure evaluation model of the user A is stored.

S503. The psychological pressure evaluation device establishes the psychological pressure evaluation model of the user A.

Specifically, the psychological pressure evaluation device collects a physiological parameter reference value of the user A. and establishes the psychological pressure evaluation model of the user A based on the common psychological pressure evaluation model and the common psychological pressure evaluation model.

The psychological pressure evaluation device stores the established psychological pressure evaluation model of the user A in the memory, and continues to perform S504.

S504. The psychological pressure evaluation device measures a physiological parameter of the user A, to obtain a physiological parameter measurement value of the user A.

S505. The psychological pressure evaluation device determines a psychological pressure value change amount of the user A based on the physiological parameter measurement value of the user A and the psychological pressure evaluation model of the user A.

S506. The psychological pressure evaluation device displays the psychological pressure value change amount of the user A on a display.

Further, if the psychological pressure value change amount of the user A is greater than a second preset threshold, the psychological pressure evaluation device sends a prompt to the user A, to prompt the user A to pay attention to pressure release. The user A may further view historical data of the psychological pressure value change amount, and adjusts a lifestyle based on a change trend of the psychological pressure value change amount.

Therefore, according to the method, the psychological pressure evaluation device can perform long-term continuous, non-perceptive, and real-time psychological pressure evaluation on the user, and prompts the user in a timely manner when pressure is high, so that the user can adjust the psychological pressure in a timely manner when no body damage is caused by the psychological pressure, and use a proper pressure-release means based on a long-term change status of the psychological pressure, to keep healthy.

In addition, the method provided in this application may also be used for psychological evaluation other than psychological pressure, for example, fatigue and an emotion change.

A person skilled in the art should understand that the embodiments of this application may be provided as a method, a system, or a computer program product. Therefore, the embodiments of this application may use a form of hardware only embodiments, software only embodiments, or embodiments with a combination of software and hardware. In addition, the embodiments of this application may use a form of a computer program product that is implemented on one or more computer-usable storage media (including but not limited to a disk memory, a CD-ROM, an optical memory, and the like) that include computer-usable program code.

The embodiments of this application are described with reference to the flowcharts and/or block diagrams of the method, the device (system), and the computer program product according to the embodiments of this application. It should be understood that computer program instructions may be used to implement each process and/or each block in the flowcharts and/or the block diagrams and a combination of a process and/or a block in the flowcharts and/or the block diagrams. These computer program instructions may be provided for a general-purpose computer, a dedicated computer, an embedded processor, or a processor of another programmable data processing device to generate a machine, so that the instructions executed by a computer or a processor of another programmable data processing device generate an apparatus for implementing a specific function in one or more processes in the flowcharts and/or in one or more blocks in the block diagrams.

These computer program instructions may also be stored in a computer-readable memory that can instruct the computer or the another programmable data processing device to work in a specific manner, so that the instructions stored in the computer-readable memory generate an artifact that includes an instruction apparatus. The instruction apparatus implements a specific function in one or more processes in the flowcharts and/or in one or more blocks in the block diagrams.

These computer program instructions may also be loaded onto a computer or another programmable data processing device, so that a series of operations and steps are performed on the computer or the another programmable device, to generate computer-implemented processing. Therefore, the instructions executed on the computer or the another programmable device provide steps for implementing a specific function in one or more processes in the flowcharts and/or in one or more blocks in the block diagrams.

It is clear that, a person skilled in the art can make various modifications and variations to the embodiments of this application without departing from the spirit and scope of this application. This application is also intended to cover these modifications and variations to the embodiments of this application provided that they fall within the scope of protection defined by the following claims of this application and their equivalent technologies.

1.-14. (canceled)

15. A method, comprising:
obtaining a physiological parameter measurement value of a user;
determining a psychological pressure evaluation result of the user based on the physiological parameter measurement value of the user and a psychological pressure evaluation model of the user, wherein the psychological pressure evaluation model of the user is based on a physiological parameter reference value of the user and a common psychological pressure evaluation model, wherein the common psychological pressure evaluation model reflects a change relationship between a psychological pressure value change amount and a physiological parameter value change amount, wherein the physiological parameter value change amount is a difference between a physiological parameter measurement value and a physiological parameter reference value, and wherein the psychological pressure value change amount is a difference between a psychological pressure value corresponding to the physiological parameter measurement value and a psychological pressure value corresponding to the physiological parameter reference value; and
storing the psychological pressure evaluation result of the user.

16. The method of claim 15, further comprising obtaining the common psychological pressure evaluation model based on a training data set using a preset machine learning method, wherein the training data set comprises a plurality of training data groups, wherein an i^{th} training data group comprises an i^{th} physiological parameter value change amount and an i^{th} psychological pressure value change amount, wherein the i^{th} physiological parameter value change amount is a difference between a physiological parameter measurement value of an i^{th} tester and a physiological parameter reference value of the i^{th} tester, and wherein the i^{th} psychological pressure value change amount is a difference between a psychological pressure value corresponding to the physiological parameter measurement value of the i^{th} tester and a psychological pressure value corresponding to the physiological parameter reference value of the i^{th} tester.

17. The method of claim 15, further comprising:
determining, based on the physiological parameter measurement value of the user and the psychological pressure evaluation model of the user, a psychological pressure value corresponding to the physiological parameter measurement value of the user, wherein the psychological pressure evaluation model of the user is based on the physiological parameter reference value of the user, the psychological pressure value corresponding to the physiological parameter reference value of the user, and the common psychological pressure evaluation model.

18. The method of claim 15, further comprising, determining a psychological pressure value change amount of the user based on the physiological parameter measurement value of the user and the psychological pressure evaluation model of the user, wherein the psychological pressure evaluation model of the user is determined based on the physiological parameter reference value of the user and the common psychological pressure evaluation model.

19. The method of claim 17, wherein before obtaining the physiological parameter measurement value of the user, the method further comprises:
querying whether the psychological pressure evaluation model of the user is stored;
obtaining the physiological parameter reference value of the user when the psychological pressure evaluation model of the user is not stored;
determining the psychological pressure value corresponding to the physiological parameter reference value of the user, wherein a difference between a moment of obtaining the physiological parameter reference value of the user and a moment of determining the psychological pressure value corresponding to the physiological parameter reference value of the user is less than or equal to preset duration; and
determining the psychological pressure evaluation model of the user based on the physiological parameter reference value of the user, the psychological pressure value corresponding to the physiological parameter reference value of the user, and the common psychological pressure evaluation model.

20. The method of claim 18, wherein before obtaining the physiological parameter measurement value of the user, the method further comprises:
querying whether the psychological pressure evaluation model of the user is stored;
obtaining the physiological parameter reference value of the user when the psychological pressure evaluation model of the user is not stored; and
determining the psychological pressure evaluation model of the user based on the physiological parameter reference value of the user and the common psychological pressure evaluation model.

21. The method of claim 15, wherein the psychological pressure value corresponding to the physiological parameter reference value of the user is a subjective psychological pressure self-evaluation value of the user.

22. An electronic device, comprising:
a non-transitory memory comprising instructions; and
a processor coupled to the non-transitory memory, wherein the instructions, when executed by the processor, cause the electronic device to:
obtain a physiological parameter measurement value of a user;
determine a psychological pressure evaluation result of the user based on the physiological parameter measurement value of the user and a psychological pressure evaluation model of the user, wherein the psychological pressure evaluation model of the user is based on a physiological parameter reference value of the user and a common psychological pressure evaluation model, wherein the common psychological pressure evaluation model reflects a change relationship between a psychological pressure value change amount and a physiological parameter value change amount, wherein the physiological parameter value change amount is a difference between a physiological parameter measurement value and a physiological parameter reference value, and wherein the psychological pressure value change amount is a difference between a psychological pressure value corresponding to the physiological parameter measurement value and a psychological pressure value corresponding to the physiological parameter reference value; and
store the psychological pressure evaluation result of the user in the device.

23. The electronic device of claim 22, wherein the instructions further cause the electronic device to obtain the common psychological pressure evaluation model based on a training data set using a preset machine learning method, wherein the training data set comprises a plurality of training data groups, wherein an i^{th} training data group comprises an i^{th} physiological parameter value change amount and an i^{th} psychological pressure value change amount, wherein the i^{th} physiological parameter value change amount is a difference between a physiological parameter measurement value of an i^{th} tester and a physiological parameter reference value of the i^{th} tester, and wherein the i^{th} psychological pressure value change amount is a difference between a psychological pressure value corresponding to the physiological parameter measurement value of the i^{th} tester and a psychological pressure value corresponding to the physiological parameter reference value of the i^{th} tester.

24. The electronic device of claim 22, wherein the instructions further cause the electronic device to:
determine, based on the physiological parameter measurement value of the user and the psychological pressure evaluation model of the user, a psychological pressure value corresponding to the physiological parameter measurement value of the user, wherein the psychological pressure evaluation model of the user is based on the physiological parameter reference value of the user, the psychological pressure value corresponding to the physiological parameter reference value of the user, and the common psychological pressure evaluation model.

25. The electronic device of claim 22, wherein the instructions further cause the electronic device to:
determine, a psychological pressure value change amount of the user based on the physiological parameter measurement value of the user and the psychological pressure evaluation model of the user, wherein the psychological pressure evaluation model of the user is based on the physiological parameter reference value of the user and the common psychological pressure evaluation model.

26. The electronic device of claim 24, wherein the instructions further cause the electronic device to:
query whether the psychological pressure evaluation model of the user is stored;
obtain the physiological parameter reference value of the user when the psychological pressure evaluation model of the user is not stored;
determine the psychological pressure value corresponding to the physiological parameter reference value of the user, wherein a difference between a moment of obtaining the physiological parameter reference value of the user and a moment of determining the psychological pressure value corresponding to the physiological parameter reference value of the user is less than or equal to preset duration; and
determine the psychological pressure evaluation model of the user based on the physiological parameter reference value of the user, the psychological pressure value corresponding to the physiological parameter reference value of the user, and the common psychological pressure evaluation model.

27. The electronic device of claim 25, wherein the instructions further cause the electronic device to:
query whether the psychological pressure evaluation model of the user is stored;
obtain the physiological parameter reference value of the user when the psychological pressure evaluation model of the user is not stored; and
determine the psychological pressure evaluation model of the user based on the physiological parameter reference value of the user and the common psychological pressure evaluation model.

28. The electronic device of claim 22, wherein the psychological pressure value corresponding to the physiological parameter reference value of the user is a subjective psychological pressure self-evaluation value of the user.

29. A computer program product comprising computer-executable instructions for storage on a non-transitory computer-readable medium that, when executed by a processor, cause an apparatus to:
obtain a physiological parameter measurement value of a user;
determine a psychological pressure evaluation result of the user based on the physiological parameter measurement value of the user and a psychological pressure evaluation model of the user, wherein the psychological pressure evaluation model of the user is based on a physiological parameter reference value of the user and a common psychological pressure evaluation model, wherein the common psychological pressure evaluation model reflects a change relationship between a psychological pressure value change amount and a physiological parameter value change amount, wherein the physiological parameter value change amount is a difference between a physiological parameter measurement value and a physiological parameter reference value, and wherein the psychological pressure value change amount is a difference between a psychological pressure value corresponding to the physiological parameter measurement value and a psychological pressure value corresponding to the physiological parameter reference value; and
store the psychological pressure evaluation result of the user in the apparatus.

30. The computer program product of claim 29, wherein the computer-executable instructions further cause the apparatus to obtain the common psychological pressure evaluation model based on a training data set using a preset machine learning method, wherein the training data set comprises a plurality of training data groups, wherein an i^{th} training data group comprises an i^{th} physiological parameter value change amount and an i^{th} psychological pressure value change amount, wherein the i^{th} physiological parameter value change amount is a difference between a physiological parameter measurement value of an i^{th} tester and a physiological parameter reference value of the i^{th} tester, and wherein the i^{th} psychological pressure value change amount is a difference between a psychological pressure value corresponding to the physiological parameter measurement value of the i^{th} tester and a psychological pressure value corresponding to the physiological parameter reference value of the i^{th} tester.

31. The computer program product of claim 29, wherein the computer-executable instructions further cause the apparatus to:
determining, based on the physiological parameter measurement value of the user and the psychological pressure evaluation model of the user, a psychological pressure value corresponding to the physiological parameter measurement value of the user, wherein the psychological pressure evaluation model of the user is based on the physiological parameter reference value of the user, the psychological pressure value corresponding to the physiological parameter reference value of the user, and the common psychological pressure evaluation model.

32. The computer program product of claim 29, wherein the computer-executable instructions further cause the apparatus to:
determine a psychological pressure value change amount of the user based on the physiological parameter measurement value of the user and the psychological pressure evaluation model of the user, wherein the psychological pressure evaluation model of the user is based on the physiological parameter reference value of the user and the common psychological pressure evaluation model.

33. The computer program product of claim 31, wherein the computer-executable instructions further cause the apparatus to:
query whether the psychological pressure evaluation model of the user is stored;
obtain the physiological parameter reference value of the user when the psychological pressure evaluation model of the user is not stored,
determine the psychological pressure value corresponding to the physiological parameter reference value of the user, wherein a difference between a moment of obtaining the physiological parameter reference value of the user and a moment of determining the psychological pressure value corresponding to the physiological parameter reference value of the user is less than or equal to preset duration; and
determine the psychological pressure evaluation model of the user based on the physiological parameter reference value of the user, the psychological pressure value corresponding to the physiological parameter reference value of the user, and the common psychological pressure evaluation model.

34. The computer program product of claim 32, wherein the computer-executable instructions further cause the apparatus to:
query whether the psychological pressure evaluation model of the user is stored;
obtain the physiological parameter reference value of the user when the psychological pressure evaluation model of the user is not stored; and
determine the psychological pressure evaluation model of the user based on the physiological parameter reference value of the user and the common psychological pressure evaluation model.
